(19) United States
(12) Patent Application Publication
(10) Pub. No.: US 2007/0065336 A1
(43) Pub. Date: Mar. 22, 2007

(54) METHOD FOR STORING FOOD DURING FUMIGATION

(76) Inventors: Rudolf H. Scheffrahn, Plantation, FL (US); Jeffrey Edwards, Plantation, FL (US)

Correspondence Address: MALIN HALEY AND DIMAGGIO, PA, 1936 S ANDREWS AVENUE, FORT LAUDERDALE, FL, 33316, US

(21) Appl. No.: 11/162,671

(22) Filed: Sep. 19, 2005

(51) Int. Cl. A61L 2/20

(52) U.S. Cl. 422/32; 422/40

(57) ABSTRACT

A method using a single known nylon polymer film bag for storing and protecting food items and medications from contamination by harmful chemical residues that may be deposited during pest control fumigation of a dwelling. The method includes the steps of selecting a particular nylon polymer film bag, manually placing the food items and medications to be protected inside said plastic bag, sealing the bag to prevent entry of the gaseous chemicals used in the fumigation process, and fumigating the dwelling with one or more bags left in the dwelling during fumigation. Food items can be safely stored for later consumption inside the home or building being fumigated, thereby eliminating the need to discard or remove food from a structure that is to be fumigated.

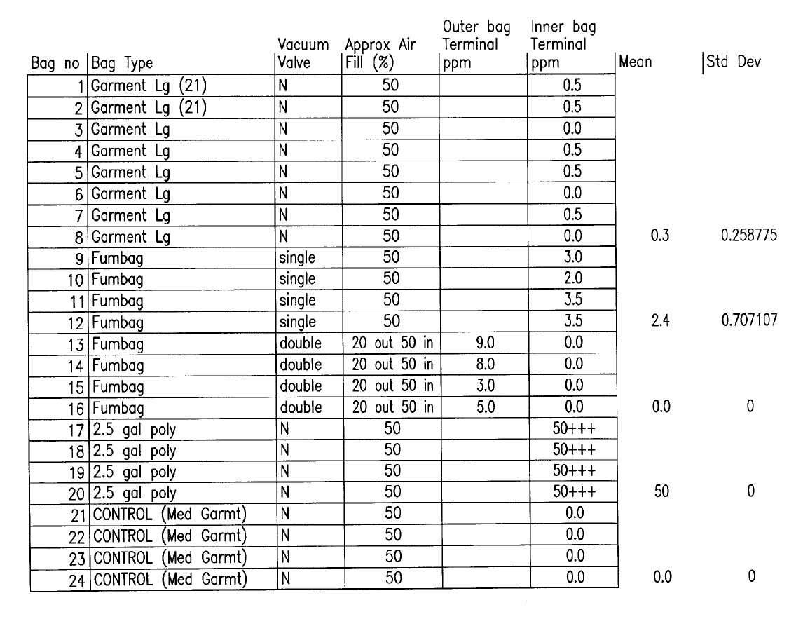

### FIELD OF THE INVENTION

This invention relates to a method for safely storing food and medications during fumigation of a building to exterminate insects and other pests so as to prevent contamination of the food and medications with harmful chemicals used in the fumigation process which can linger as residues on the food and medications.

### DESCRIPTION OF RELATED ART

The fumigation of houses and buildings to eliminate pests, such as termites, has always presented a problem with respect to food and medicine storage. Structural fumigation with sulfuryl fluoride (SF) and methyl bromide (MB) has been practiced for decades to control dry wood termites, wood-boring beetles, and various miscellaneous structural and household pests not easily controlled by other, less-demanding, methods. The resident pest population is eradicated because fumigation exposes the entire structure and all of its contents, including wood matrices, to lethal concentrations of the gaseous toxin. Label direction for SF and MB dictate that foods, feed, and medicines must either be properly protected from fumigant exposure (e.g., sealed metal or glass containers or, on older SF labels, polyethylene bags) or should be removed from the structure because unprotected commodities are likely to harbor transient or permanent residues of either SF or MB after fumigation. Many consumer foods packaged in manufacturer-sealed containers of various materials and closure types are also susceptible to fumigant exposure and residue formation, and often the packaging does not constitute adequate protection. The removal of foods from a house or commercial building before fumigation of the structure is often impractical or even unfeasible. Conventional practice is to retain the foods in the house being fumigated by the use of double bagging of bags made of nylon film. In double bagging, a first bag is used to receive the individual food items. The bag is banded or sealed and then a second bag is used for the insertion of the first sealed bag. Double bagging has been proven effective in preventing contamination of food products. However, the act of double bagging numerous food items in a dwelling before the fumigation process is labor intensive. The U.S. Environmental Protection Agency requires that all food and medicinal items, including refrigerated and frozen foods, either must be removed from the structure or double bagged due to the potential for chemical residues. Double bagging (two nylon bags, one inside another) has required manual insertion of the food stuffs and medications, hand tying the first bag and placing the bag with the food stuff inside another bag and hand tying. Labor intensive manual work is involved in fumigation of a house or commercial establishment and protecting food and medicinal products.

Sealable nylon bags are well known in the prior art; however, the prior art does not include any methods for storing foods safely in sealed plastic bags during the fumigation of a home or building. Only double bagging has been acceptable.

U.S. Pat. No. 3,502,487, issued to Byrd on Mar. 24, 1970, describes a food preserving package and method of closure, which includes vents for releasing excess vapors and fluids from the package to accommodate extreme temperatures and pressures. The '487 patent does not disclose a method for storing and protecting food items from the chemicals used during pest control fumigation. Similarly, U.S. Patent Application Publication No. US 2004/0170851, published by Lischefski on Sep. 2, 2004, describes a packaging film containing coextruded polyester and nylon layers that is useful for packaging both food and non-food items. The Lischefski patent application does not describe a method for storing and protecting food during pest control fumigation with the claimed packaging film.

U.S. Pat. No. 4,532,652, issued to Herrington on Jul. 30, 1985, U.S. Pat. No. 4,834,554, issued to Stetler et al., on May 30, 1989, U.S. Pat. No. 5,332,095, issued to Wu on Jul. 26, 1994, U.S. Pat. No. 5,996,800, issued to Pratt on Dec. 7, 1999, U.S. Pat. No. 6,059,457, issued to Sprehe et al. on May 9, 2000, U.S. Pat. No. 6,116,781, issued to Skeens on Sep. 12, 2000, and U.S. Pat. No. 6,491,433, issued to Shabram et al., on Dec. 10, 2002, all disclose plastic bags with venting means or valves. The '457 patent includes a valve for evacuating air from a zipper-sealed bag, and is used primarily for evacuating air from clothing inside a bag to preserve the clothing from degradation by bacteria and other microorganisms. Similarly, the '095 patent claims an air-impermeable bag with a valve for vacuum sealing food inside to prevent the entrance of moisture that would degrade the freshness and edibility of said food. U.S. Patent Application Publication No. US 2004/0188310, published by Hamilton et al., on Sep. 30, 2004, also describes a storage bag having a venting structure for removing air from the bag and a seal for preventing air from re-entering said bag. The valve and vent structures in all of these patents and patent applications are for releasing or removing oxygen from the bag to prevent the growth of bacteria which may degrade the contents, particularly food items, stored within the bag. However, none of these patents or published patent applications teaches a method for using a sealable plastic bag to store and protect food from contamination by chemicals released during pest control fumigation.

U.S. Pat. No. 5,142,970, issued to ErkenBrack on Sep. 1, 1992, describes an apparatus for storing matter, and particularly food items, out of contact with gas. The '970 patent claims a vacuum pump that can be connected to the wall of a container for exhausting air from said container to store the contents of said container out of contact with gas. The '970 invention does not claim any method for using vacuum-sealed bags or containers to prevent contamination of food items by contact with fumigation gases used in pest control. U.S. Patent Application Publication No. US 2003/0224091, published by Nayyeri on Dec. 4, 2003, describes a bag for storing rice and cereals to prevent insect infestations of the food. U.S. Patent Application Publication No. US 2004/0007494, published by Popeil et al., on Jan. 15, 2004, describes an apparatus and method for effectively vacuum-packaging foods. Neither of these applications describes a method for storing food during fumigation to prevent contamination by pest control chemicals.

Finally, U.S. Pat. No. 6,033,113, issued to Anderson on Mar. 7, 2000, describes a seal for zipper-type plastic bags, but does not teach a method for storing and protecting food within a zipper-sealed bag during pest control fumigation to prevent contamination of said food items. New West Products, Inc. of San Diego, Calif. manufactures and sells a space saver plastic bag that includes a seal for zipper-type plastic bags in accordance with the invention claims in U.S. Pat. No. 6,033,113. The New West plastic bags are sold under the trademarks of “space bag” and “space savers.”

A single sealable bag, in lieu of double bagging is a way to greatly reduce the cost of house fumigation to preserve food and medicinals.

The present invention uses a single nylon bag (without a vent) that can be closed with an airtight seal often referenced to as a zip lock seal for storing food items and medications.

Applicants have found a method of using a sealable nylon bag for protecting food during domestic fumigation that is safe and less labor intensive.

### SUMMARY OF THE INVENTION

A method for storing and protecting foods and medications to prevent contamination by chemical residues introduced in a home or building during pest control fumigation that employs a single sealable nylon bag. Said method comprises the steps of placing food items and medications inside a particular existing fumigant impermeable bag for storage, and then, sealing said bag closed to prevent entry of gaseous contaminants produced by fumigation of a home or building for pest control purposes. A single bag is used as a protective barrier for food items and medications during fumigation by providing the greatest reduction in the amount of contaminants that come in contact with said food and other stored items with a sealable opening that can be quickly sealed manually.

In the preferred embodiment of the invention, one bag that can be used in this method is described in U.S. Pat. No. 6,033,113. The bag is comprised of a top sheet and a bottom sheet permanently bonded and closed on three sides with said bag having an airtight zipper seal on a fourth open end that receives objects placed within said bag. Said top and bottom sheets of the bag are each constructed from a layer of extruded low-density polyethylene film sandwiched between a layer of nylon sheeting and a layer of linear low-density polyethylene film. Applicants have determined that the method is safe and food stuffs are not contaminated in such a bag during fumigation.

A zipper-like seal is the preferred closure utilized with bags used in this method of storing and protecting food during pest control fumigation because of reduced labor time to close the bag. In addition to food items and medications, said method may also be used for storing and protecting cosmetics, pet foods, and other ingestible products during fumigation of a house or building to prevent contamination by chemical residues deposited by the pest control gases used during the fumigation process.

An object of this invention is to provide a safe, effective, and inexpensive method for storing and protecting food items, medications, pet foods, and other ingestible products during pest control fumigation so as to reduce the amount of harmful chemical residue that is deposited on said food items and other products in the absence of protective storage.

Another object of this invention is to eliminate labor, time and waste by safely storing food items and other ingestible products so that the products do not have to be discarded subsequent to fumigation using a single storage bag.

In accordance with these and other objects which will become apparent hereinafter, the instant invention will now be described with particular reference to the accompanying drawings.

### BRIEF DESCRIPTION OF THE DRAWING

The drawing shows performance data to determine efficacy of certain bags used in the invention.

### DETAILED DESCRIPTION

A method of using a single known garment bag for storing and protecting foods and medications to prevent contamination by chemical residues introduced in a home or building during pest control fumigation. Said method comprises the steps of, first selecting a particular known garment bag, placing food items and medications inside said particular single bag, and manually closing and sealing said bag to prevent entry of gaseous contaminants produced by fumigation of a home or building for pest control purposes. The building is then fumigated with the bag left in the building during fumigation. The specific single bag is described in U.S. Pat. No. 6,033,133 and is approximately two feet long and 18 inches wide, with a zipper-like plastic lip seal along the top opening. Different sized bags can be used. A single bag is used as a protective barrier for food items and medications during fumigation.

In the preferred embodiment of the invention, the bag used in this method is one comprised of a top sheet and a bottom sheet permanently bonded and closed on three sides with said bag having a zipper-like plastic lip seal on a fourth open end that receives food and medicants placed within said single bag. Said top and bottom sheets of the bag are each constructed from a layer of extruded low-density polyethylene film sandwiched between a layer of nylon sheeting and a layer of linear low-density polyethylene film. This type of bag is manufactured by New West Products, Inc, of San Diego, Calif., and is covered by U.S. Pat. Nos. 6,116,781 and 6,033,113. The applicants hereby incorporate by reference U.S. Pat. Nos. 6,116,781 and 6,033,113. A warning label printed on this particular bag, which is sold under license under the Coleman® trademark, teaches against storing food inside said bag, and other labels printed on the bag state that said bag is to be used for storing garments, electronics, and documents. The applicants are the first to teach using this type of bag as the preferred method for storing and protecting food in a house or other building that is to be fumigated for pest control purposes.

Zipper-like seals are the preferred means of closure utilized with a bag used in this method of storing and protecting food during pest control fumigation. In addition to food items and medications, said method may also be used for storing and protecting cosmetics, pet foods, and other ingestible commodities during fumigation of a house or building to prevent contamination by chemical residues deposited by the pest control gases used during the fumigation process.

Referring now to the drawing to determine their efficacy as a fumigant barrier, sealed bags were fumigated in a 1,000 cubic foot chamber. The experimental procedure involved placing three different bag types into a 1,000 cubic foot fumigation chamber that was fumigated with sulfuryl fluoride at a mean concentration of 2629 parts per million (ppm) for 22.5 hours at a mean temperature and relative humidity of 77 degrees F. and 73% RH. Four bag treatments include a single New West Products large garment Space Bags with Ziploc seal (bag numbers 1-8), single or double Nylofume bags (prior art) sealed by twisting the neck and securing with a rubber band (bag numbers 9-16), and Hefty 2.5 gallon polyethylene food storage bags with Ziploc seal (bag numbers 17-20). Un-fumigated control bags consisted of New West Products medium garment Space Bags with Ziploc seal (bag numbers 21-24). After the fumigation, bags were immediately removed and internal bag concentration was tested using an Interscan Gas Analyzer with maximum readout capacity of 50 ppm. The results are presented in the drawing.

The instant invention has been shown and described herein in what is considered to be the most practical and preferred embodiment. The applicants recognize, however, that departures may be made therefrom within the scope of the invention and that obvious modifications will occur to a person skilled in the art.

What is claimed is:

1. A method of using a single known plastic bag containing a nylon polymer film for storing and protecting foods and medications to prevent contamination by chemical residues introduced in a home or building during pest control fumigation, said method comprising the steps of selecting a known nylon polymer film bag that is constructed as a barrier for fumigation chemicals;
placing food items and medications inside said selected bag for storage;
sealing said bag closed to prevent entry of gaseous contaminants produced by fumigation of a home or building for pest control purposes; and
fumigation said house with chemicals with the bag kept in said house.

2. The method of claim 1, wherein the preferred bag for use in this method is one comprised of a top sheet and a bottom sheet permanently bonded and closed on three sides and having a zipper-like mechanical seal on a fourth open end which receives objects placed within said bag.

3. The method of claim 2, wherein said top and bottom sheets are each constructed from a layer of extruded low-density polyethylene film sandwiched between a layer of nylon sheeting and a layer of linear low-density polyethylene film.

4. The method of claim 1, wherein said method may also be used for storing and protecting cosmetics, pet foods, and other ingestible commodities during fumigation of a house or building to prevent contamination by chemical residues deposited by the pest control gases used during the fumigation process.

5. The method of claim 1, wherein said bag may comprise a plastic garment storage bag, a single-layer polymer film bag, and/or a double-layer polymer film bag as well as bags constructed from any other suitable material that is fumigant impermeable.

6. The method claim 1, wherein bags closed by means other than zipper seals, including, but not limited to, tape, knots, heat-seals, and twist-ties, may also be used to store and protect food items during fumigation.
